(19) United States
(12) Patent Application Publication
(10) Pub. No.: US 2006/0089408 A1
(43) Pub. Date: Apr. 27, 2006

(54) Method of treating blepharospasm with a prostaglandin derivative

(76) Inventors: Ted C. Wei JR., Monterey Park, CA (US); Ted Chau-Po Wei, Monterey Park, CA (US)

Correspondence Address: RAYMOND Y. CHAN, 108 N. YNEZ AVE., SUITE 128, MONTEREY PARK, CA, 91754, US

(21) Appl. No.: 10/971,650

(22) Filed: Oct. 22, 2004

(51) Int. Cl. A61K 31/557

(52) U.S. Cl. 514/530

(57) ABSTRACT

The present invention is a method of treating blepharospasm with a prostaglandin derivative, comprising the steps of (a) providing a predetermined amount of prostaglandin derivative; and (b) applying the prostaglandin derivative in a predetermined position. The prostaglandin derivative is preferably in the form of latanoprost wherein the chemical composition of latanoprost is a prostaglandin F.sub.2.alpha derivative which has the chemical name isopropyl-(Z)-7[(1R,2R,3R,5S)3,5-dihydroxy-2-[(3R)-3-hydroxy-5-phenylpentyl I]cyclo-pentyl]-5-heptenoate. The dosage of the prostaglandin derivative is generally in the range of 1.5 ug to 4.5 ug which is equivalent to 1 to 3 drops aqueous solution for human eye

### BACKGROUND OF THE PRESENT INVENTION

1. Field of Invention

The present invention relates to a method of treating blepharospasm with a prostaglandin derivative, and more particularly to a method of treating blepharospasm with a prostaglandin derivative preferably in the form of latanoprost which is effective and capable of providing a topical medication administered for treating blepharospasm.

2. Description of Related Arts

A human eye is surrounded by the orbicularis oculi muscle and several other muscles which are jointly called the muscles of facial expression. In describing the phenomenon of blepharospasm, there are numerous classifications based on anatomic components and subjective severity. The main muscle of focus is the orbicularis oculi muscle, innervated by the seventh cranial nerve which controls both voluntary and involuntary closure of the eyes. Blepharospasm is generally described as persistent involuntary spasms of the eyelid protractors, mainly the orbicularis oculi, corrugator supercilli and the procerus muscles that can be transient or chronic, which can produce complete closure of the eyelids and contraction of surrounding muscles.

A patient suffering Blepharospasm generally has a normal eye and the visual disturbance is mainly the complete closure of the eyelids which block eyesight. Initial symptoms can be episodic and minor in nature. Eventually, one may experience increased blinking, ocular irritation, sensation of ocular dryness, light sensitivity and, if chronic, can cause complete closure of the eye. Many patients who suffer from blepharospasm do not mention their symptoms because they have generally adapted their lifestyle to accommodate these symptoms.

The cause of primary blepharospasm is unknown and presumed to be multifactorial. One of the causes may be due to the abnormal functioning of the basal ganglia. Blepharospasm is capable of being classified into several categories mainly by severity. One of the classifications, as described by Anderson (Anderson, R. L. et al. Blepharospasm: Past, present and future. Ophthal Plast Resonstr Surg 1998; 14:305-317), includes the categories of blepharospasm, Meige Syndrome, Brueghee's syndrome, segmental cranial dystonia, and generalized dystonia according to an ascending order of severity.

There are currently three modalities of treatment that exist for chronic blepharospasm, namely, neurological treatment involving oral centrally acting medication, Botulinum toxin injections, and surgical treatment, in addition to the education and support through the Benign Essential Blepharospasm Research Foundation.

Neurological treatment involves medication having a variety of mechanisms of actions and it is difficult to have satisfactory control. Drugs containing trihexyphenidyl, benztropine, diazepam, clonazepam, baclofen, carbamazepine, levodopa, methyldopa, bromocriptime, amantadine, tetrabenazine and others may be used as medication and their effects may be different for different patients. Furthermore, undesirable side effects and unpredictable results may be induced with the use of these medications. The overall efficacy of medication is not satisfactory.

Botulinum toxin type A was first introduced in 1989 wherein the appropriate concentration is injected into the eyelid superficial to the pretarsal orbicularis. At present, botulinum toxin injection is still the most common form of treatment. The injection is capable of weakening the muscles of the eye responsible for eye spasm and hence mitigating the involuntary movement of the muscles which lead to eye closure. The injection may also indirectly influence the oculomotor control of the central nervous system by altering the input from motor nerve afferents. However, though the results of the botulinum toxin injection are more satisfactory than that of oral medications, the overall efficacy of botulinum toxin injection is still not sufficiently satisfactory. Moreover, undesirable side effects may be induced and the effect of each injection is temporary and requires repetitive injections every three to six months.

When medication and botulinum toxin injection are not capable of relieving the symptom of Blepharospasm, surgical treatment may be applied as a final option. The treatment is surgical in nature including myectomy which involves surgically identifying the orbicularis oculi muscle and severing its nerves, thus inherits the inherent risk of surgical operation. Other cosmetic procedures may also be included in the surgical treatment for the patient, but the substantive inherent risk of surgical operation could not be eliminated.

As we may see from the above three conventional methods of treatment for Blepharospasm are both unsatisfactory and undesirable, there is really a need to look for a more prospective, non invasive and effective treatment to provide a relief for patients having normal eye but Blepharospasm.

Prostaglandin derivative is the major composition of the present invention which has been widely used for some time for the treatment of ocular hypertension and glaucoma. Ocular hypertension is a condition of elevated intraocular pressure which is generally believed to be an earliest phase of glaucoma while glaucoma is a group of diseases that display a certain pattern of damage to the optic nerve which can result in blindness. Prostaglandin and prostaglandin derivatives are capable of lowering intraocular pressure by increasing uveoscleral outflow in the eye and thus decreasing the intraocular pressure.

Prostaglandin and its derivatives are also used in other treatments. For example, the U.S. Pat. No. 5,905,091 disclosed the enhancement of skin pigmentation by prostaglandins, the U.S. Pat. No. 6,262,105 disclosed the method of enhancing hair growth with the use of prostaglandins, the U.S. Pat. No. 6,225,348 disclosed the method of treating macular degeneration with a prostaglandin derivative, the U.S. Pat. No. 6,225,348 disclosed the method of treating macular degeneration with a prostaglandin derivative. However, no studies or work of prostaglandin and its derivatives has been done in the treatment of Blepharospasm.

### SUMMARY OF THE PRESENT INVENTION

A main object of the present invention is to provide a method of treating blepharospasm with a prostaglandin derivative.

Another object of the present invention is to provide a method of treating blepharospasm with a prostaglandin derivative preferably in the form of latanoprost with a preferred dosage of 1.5 ug.

Another object of the present invention is to provide a method of treating blepharospasm with a prostaglandin derivative for relieving some forms of blepharospasm.

Another object of the present invention is to provide a method of treating blepharospasm with a prostaglandin derivation wherein periodical injection treatment is avoided.

Another object of the present invention is to provide a method of treating blepharospasm with a prostaglandin derivative which is satisfactory and effective and is capable of administering in the form of topical solution.

Another object of the present invention is to provide a method of treating blepharospasm with a prostaglandin derivative which is satisfactory and effective for relieving some forms of blepharospasm.

Another object of the present invention is to provide a method of treating blepharospasm with a prostaglandin derivative which is easy and convenience to be applied.

Accordingly, in order to accomplish the above objects, the present invention is a process of treating blepharospasm with a prostaglandin derivative comprising the step of applying the prostaglandin derivative in a predetermined position.

The prostaglandin derivative is preferably in the form of latanoprost wherein the chemical composition of latanoprost is a prostaglandin F.sub.2.alpha derivative which has the chemical name isopropyl-(Z)-7[(1R,2R,3R,5S)3,5-dihydroxy-2-[(3R)-3-hydroxy-5-phenylpentyl I]cyclo-pentyl]-5-heptenoate.

These and other objectives, features, and advantages of the present invention will become apparent from the following detailed description, the accompanying drawings, and the appended claims.

### DETAILED DESCRIPTION OF THE PREFERRED EMBODIMENT

The present invention is a method of treating blepharospasm with a prostaglandin derivative with a prostaglandin derivative comprising the step of applying a predetermined dosage of the prostaglandin derivative in a predetermined position wherein the prostaglandin derivative is preferably in the form of latanoprost have a chemical composition prostaglandin F.sub.2.alpha derivative which has the chemical name isopropyl-(Z)-7[(1R,2R,3R,5S)3,5-dihydroxy-2-[(3R)-3-hydroxy-5-phenylpentyl]cyclopentyl]-5 -heptenoate.

The dosage of the prostaglandin derivative is generally in the range of 1.5 ug to 4.5 ug which is equivalent to 1 to 3 drops aqueous solution for human eye.

It is the inventor's unique discovery that latanoprost improves some forms of blepharospasm. Over a period of three years starting from 2000, 21 patients with various forms of blepharospasm were treated with latanoprost. In more than 75% of the cases observed, the symptoms of blepharospasm were much improved ranging from mild resolution to complete improvement.

Observational prospective case series of 21 consecutive patients comprising mostly Asian descent with blepharospasm were treated with Latanoprost for a mean follow-up period of 1.5 years. Assessment of therapy was made by symptomatic evaluation and clinical examination. Severity of patient's initial symptoms ranged from myokimia to blepharospasm with facial dystonia. Clinical results showed that over 75% of patients reported moderate to complete resolution of symptoms after treatment with Latanoprost.

The patients were pooled from the Los Angeles County metropolitan area and the Pacific Rim and all of the patients are of Chinese descent. The patients were excluded if they had secondary causes of blepharospasm such as light sensitivity, lid hygiene (blepharitis), dry eyes, and emotional or physical psychological stressors.

Therapeutic intervention involved administration of one drop of Latanoprost in the affected eye at night. Adverse effects were assessed by the patient's subjective feedback and physician's examination. Outcome assessment was determined mainly by subjective reporting by the patient.

Physician's Grading Scale was adopted in Table 1 as follows:

TABLE 1
Physician's Grading Scale

| Grade | Description |
|---|---|
| I | Blepharospasm |
| II | Blepharospasm with associated facial |
| | dystonia |
| III | Blepharospasm with facial dystonia |
| | extending to neck |

Table 1 illustrates the physician's grading scale, wherein grade I signified blepharospasm isolated just to the eyes; Grade II described blepharospasm with some form of facial dystonia; and Grade III described blepharospasm with facial dystonia extending to the neck region

Patient's subjective evaluation of improvement was graded in Table 2 as follows:

TABLE 2
Patient's subjective Evaluation Grading

| Grade | Description |
|---|---|
| 0 | No improvement |
| 5 | 50% improvement |
| 10 | Complete resolution |

Table 2 illustrates patient's subjective evaluation of improvement that subjective opinion of improvement is on a scale ranging from 0 to 10, where zero indicated no improvement and 10 indicated complete resolution.

TABLE 3
Demographics and Clinical Data

| | Name | Age | Sex | Duration | | Eye | Grade | Date start | Improvement | | Scale imp |
|---|---|---|---|---|---|---|---|---|---|---|---|
| 1 | KL | 82 | F | 18 | yr | OS | II | Jun-00 | 2 | months | 9 |
| 2 | EY | 86 | M | 35 | yr | OD | II | Jul-00 | 2 | months | 8 |
| 3 | HC | 70 | F | 4 | yr | OD | II | Sep-00 | 1 | month | 8 |
| 4 | KL | 78 | F | 10 | yr | OD | II | Nov-00 | 2 | months | 5 |
| 5 | TL | 45 | F | 1 | mo | OS | I | Jan-01 | 2 | months | 10 |
| 6 | PC | 76 | M | 9 | yr | OS | II | Jan-01 | 2 | months | 5 |
| 7 | SH | 55 | F | 10 | yr | OU | I | Feb-01 | 2 | months | 10 |
| 8 | SL | 76 | M | 23 | yr | OU | II | Feb-01 | 2 | months | 8 |
| 9 | YT | 71 | F | 10 | y | OS | II | Feb-01 | 1 | month | 5 |
| 10 | NL | 76 | F | 1 | mo | OD | II | Jul-01 | 1 | month | 8 |
| 11 | YL | 72 | F | 6 | mo | OS | II | Aug-01 | 2 | months | 7 |
| 12 | KM | 78 | M | 3 | yr | OS | II | Sep-01 | 2 | months | 5 |
| 13 | PL | 71 | F | 6 | mo | OS | II | Aug-02 | 1 | month | 9 |
| 14 | JL | 65 | F | 11 | yr | OU | II | Oct-02 | 2 | months | 9 |
| 15 | SC | 48 | M | 1 | mo | OS | II | Oct-02 | 2 | months | 6 |
| 16 | KP | 74 | M | 6 | mo | OD | II | Oct-02 | | | 0 |
| 17 | ES | 74 | F | 8 | mo | OS | II | Oct-02 | 2 | months | 5 |
| 18 | ML | 68 | F | 23 | yr | OS | I | Oct-02 | 2 | months | 5 |
| 19 | TC | 51 | M | 6 | mo | OD | II | Nov-02 | | | 0 |
| 20 | WJ | 73 | M | 3 | mo | OU | I | Jan-03 | 1 | month | 8 |
| 21 | EY | 78 | M | 3 | mo | OS | I | Apr-03 | 1 | month | 8 |

I = Stage I,
II = Stage II,
III = Stage III

Table 3 illustrates demographics and clinical data of the patients involved in the above clinical study. In Table 3, F represent female, M represent male, duration represents duration of treatment, OU represents blepharospasm in both eyes, OD represents blepharospasm in right eye only, and OS represents blepharospasm in left eye. Grade represents the physician's grading scale as shown in Table 1. Date start illustrates the date starting treatment of prostaglandin derivatives, improvement illustrates the time required for first noticeable improvement, while scale imp represents scale of improvement according to the subjective evaluation grading as shown in Table 2.

The average age of the patients was 70 with a range of 45-86 years old. There were 9 males and 12 females, all of whom were of Chinese descent. Blepharospasm was noted in both eyes in 4 patients, while the remainder had unilateral blepharospasm. Six of the patients had blepharospasm in the right eye while eleven in the left eye. After treatment with Latanoprost. 19 of 21 patients noted subjective improvement in their blepharospasm and 2 patients reported no change. Noticeable improvements occurred between 1-2 months after initiation of treatment, and the average amount of improvement was 6.95. Four patients had concurrent diagnosis of glaucoma and were on other glaucoma medications before treatment.

Some of the selected cases from the above observational consecutive cases are further illustrated in the following case description.

Case 1 is a 62 years old female with a 30 years history of blepharospasm with facial dystonia in the right eye. She has been evaluated and treated by oral drugs or behavioral modification by numerous neurologists and ophthalmologists with no resolution of symptoms. Her symptoms were very severe and chronic that she required ocular crutches to keep her eye open otherwise she had functional blindness. The preferred prostaglandin derivative latanoprost was prescribed with a dosage of 1.5 ug per day. After 3 weeks of treatment she noticed considerable decrease in spasm of her eyelids and face and was able to voluntarily open her eyelids without the assistance of ocular crutches.

Case 2 is a patient who is a 86 year old Asian male and had been suffering from blepharospasm in his right eye for the past 35 years. He had previously tried oral medications and acupuncture without relief of his symptoms. Over the years, no progression or resolution of his spasm has been seen and he was not interested in botulinum injections. On Jul. 6, 2000, he started on Xalatan having the dosage of one drop in the right eye every night. After two months, he stated that his spasm had improved to grade 8. After five months treatment, he remained at 8 on the improvement scale. Then, Xalatan was discontinued and his spasm worsened in two months time, that his improvement scale went down to 5. Xalatan was restarted and his grade is improved to 9 after several months of treatment.

Case 7 is an Asian female patient of 55 years old who presented with a 10 years history of blepharospasm in the left lower lid. She had no significant past medical history and was currently on no medications. She was evaluated and started on Xalatan one drop in the left eye every night. Two months later, she reported that her blepharospasm had completely resolved and that she had used Xalatan for only one month. The spasm had not recurred since she discontinued the medication.

Case 14 is a 65 year old Asian female who presented with a complaint of forehead pain, epiphora, and mild ocular pain for a few weeks prior to presentation and a 11 year history of blepharospasm in both eyes. She had been seen by numerous specialists and tried numerous treatments including botulinum, acupuncture, and medication, and no resolution has appeared. She started Xalatan, having one drop in both eyes every night. Approximately 6 weeks later, she described an improvement of 4-5 from her baseline. After 3 month follow up, she noted an improvement of 5 OD and an improvement of 9 OS. After 4 months treatment, she noted an improvement to 9 in both eyes and she remained at this level with Xalatan medication.

Latanoprost was originally developed for the treatment of elevated intraocular pressure (IOP) in glaucoma and ocular hypertensive patients. Since its introduction several side effects have been discovered including follicular growth and melanin production which were later shown to have possible clinical applications.

In the present invention, a strong relationship between the use of Latanoprost and the resolution of symptoms of blepharospasm is observed. As with elevated intraocular pressure studies with Latanoprost, approximately 2 months of treatment were required before its effects were seen.

There may also be a relationship between a patient's ethnic background and the efficacy of Latanoprost. A recent study by Hedman (Hedman, K., Larsson, L. The Effect of Latanoprost Compared with Timolol in African-American, Asian, Caucasion, and Mexican Open-Angle Glaucoma or Ocular Hypertensive Patients Survey of Ophthalmology Supp 1 2002; S77-S89) pointed out there was a significant difference in efficacy between Asian/Hispanic patients and Caucasian/African patients when using Latanoprost to treat glaucoma. Since all our patients were Asian, further studies need to be done in other ethnic groups to evaluate if this hypothesis also applies to the treatment of blepharospasm in other ethnic groups.

It is shown that although some of our patients experienced complete resolution which persisted even after discontinuing the medication, others required continued application for continued relief of blepharospasm.

One patient's progress deteriorated after experiencing a devastating divorce, then recovered after several months. This observation supports the theory that blepharospasm may be multi-factorial in nature, including a strong psychosomatic component.

According to the observational series of the present invention, a preferred embodiment of the present invention is a method of treating blepharospasm with a prostaglandin derivative, comprising the steps of:

(a) providing a predetermined amount of prostaglandin derivative; and

(b) applying the prostaglandin derivative in a predetermined position.

The prostaglandin derivative is preferably in the form of latanoprost wherein the chemical composition of latanoprost is a prostaglandin F.sub.2.alpha derivative which has the chemical name isopropyl-(Z)-7[(1R,2R,3R,5S)3,5-dihydroxy-2-[(3R)-3-hydroxy-5-phenylpentyl I]cyclo-pentyl]-5-heptenoate.

The dosage of the prostaglandin derivative is generally in the range of 1.5 ug to 4.5 ug which is equivalent to 1 to 3 drops aqueous solution for human eye.

According to the preferred embodiment of the present invention, the method of treating blepharospasm with a prostaglandin derivative may further comprise the steps of:

(c) providing a standard evaluation grading reference with respect to improvement of blepharospasm; and

(d) monitoring an evaluation grading of a user according to the standard evaluation grading reference.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure form such principles. Therefore, this invention includes all modifications encompassed within the spirit and scope of the following claims.

What is claimed is:

1. A method of treating blepharospasm comprising the steps of:
(a) providing a predetermined amount of prostaglandin derivative; and
(b) administering the prostaglandin derivative in a predetermined position.

2. The method, as recited in claim 1, wherein the prostaglandin derivative is latanoprost.

3. The method, as recited in claim 1, wherein the prostaglandin derivative is a prostaglandin F.sub.2.alpha derivative having a chemical name isopropyl-(Z)-7[(1R,2R,3R,5S)3,5-dihydroxy-2-[(3R)-3-hydroxy-5-phenylpentyl I]cyclo-pentyl]-5-heptenoate.

4. The method, as recited in claim 1, wherein the prostaglandin is administered by employing topical administration.

5. The method, as recited in claim 3, wherein the prostaglandin is administered by employing topical administration.

6. The method, as recited in claim 3, wherein the predetermined position is an area of an affected eye suffered from blepharospasm.

7. The method, as recited in claim 5, wherein the predetermined position is an area of an affected eye suffered from blepharospasm.

8. The method, as recited in claim 3, wherein the predetermined amount of prostaglandin derivative is in a range between 1.5 ug and 4.5 ug.

9. The method, as recited in claim 5, wherein the predetermined amount of prostaglandin derivative is in a range between 1.5 ug and 4.5 ug.

10. The method, as recited in claim 3, wherein the predetermined amount of prostaglandin derivative is in a range between 1 and 3 drops aqueous solution of the prostaglandin derivative.

11. The method, as recited in claim 5, wherein the predetermined amount of prostaglandin derivative is in a range between 1 and 3 drops aqueous solution of the prostaglandin derivative.

12. The method, as recited in claim 1, further comprising the steps of
(c) providing a standard evaluation grading reference for quantifying improvement; and
(d) monitoring a condition of an user according to the condition of the user.

13. The method, as recited in claim 12, wherein the standard evaluation grading reference is in a range between zero (0) to ten (10), wherein 0 represents no improvement, 5 represents 50% improvement, and 10 represents complete resolution.

14. The method, as recited in claim 5, further comprising the steps of
(c) providing a standard evaluation grading reference for quantifying improvement; and
(d) monitoring a condition of an user according to the condition of the user.

15. The method, as recited in claim 14, wherein the standard evaluation grading reference is in a range between zero (0) to ten (10), wherein 0 represents no improvement, 5 represents 50% improvement, and 10 represents complete resolution.

16. The method, as recited in claim 7, further comprising the steps of
(c) providing a standard evaluation grading reference for quantifying improvement; and
(d) monitoring a condition of an user according to the condition of the user.

17. The method, as recited in claim 16, wherein the standard evaluation grading reference is in a range between zero (0) to ten (10), wherein 0 represents no improvement, 5 represents 50% improvement, and 10 represents complete resolution.

18. The method, as recited in claim 9, further comprising the steps of
(c) providing a standard evaluation grading reference for quantifying improvement; and
(d) monitoring a condition of an user according to the condition of the user.

19. The method, as recited in claim 18, wherein the standard evaluation grading reference is in a range between zero (0) to ten (10), wherein 0 represents no improvement, 5 represents 50% improvement, and 10 represents complete resolution.
